(19) United States
(12) Patent Application Publication
(10) Pub. No.: US 2009/0209830 A1
(43) Pub. Date: Aug. 20, 2009

(54) METHOD AND APPARATUS FOR MONITORING EXTERNAL PHYSICAL PARAMETERS HAVING AN INFLUENCE ON THE ONSET OR PROGRESSION OF A MEDICAL CONDITION

(76) Inventors: Richard Nagle, London (GB); Andrea Moretti, London (GB)

Correspondence Address: DICKSTEIN SHAPIRO LLP, 1825 EYE STREET NW, Washington, DC, 20006-5403, US

(21) Appl. No.: 12/307,500

(22) PCT Filed: May 21, 2007
(86) PCT No.: PCT/GB07/01842

§ 371 Date: Jan. 15, 2009

(30) Foreign Application Priority Data
Jul. 6, 2006 (GB) 0613464.7

(51) Int. Cl. A61B 5/00; A61F 5/00

(52) U.S. Cl. 600/301; 602/2

(57) ABSTRACT

A patient compliance monitor (10) for monitoring compliance of a patient to a treatment regime for treatment of a medical condition, the patient compliance monitor comprising measurement means (12) for measuring an external physical parameter acting on a limb of the said patient, the external physical parameter having an influence on the medical condition experienced by said limb; recording means (14) for recording over a period of time data representative of the physical parameter; and comparative means for comparing the recorded data with data indicative of the treatment regime to determine patient compliance to the treatment regime.

The present invention relates to a method and apparatus for monitoring external physical parameters having an influence on the onset or progression of a medical condition. The invention also relates to a method of and an apparatus for monitoring treatment of a wound and compliance of a patient to a treatment regime.

### BACKGROUND

The onset or progression of medical conditions such as, for example, DVT or chronic wounds may be influenced by external physical parameters. For example physical activity such as walking or leg elevation can for example have a beneficial effect on the prevention of DVT or the healing of a chronic wound.

A deep vein thrombosis (DVT) is a blood clot (thrombus) that develops in a deep vein, usually in the lower leg. Deep vein thrombosis can cause pain in the leg and can potentially lead to complications, the most serious of which is the life-threatening condition of pulmonary embolism (60,000 people die in the US every year from embolism). Risk factors for developing DVT include obesity, family history, conditions which cause immobility like hip surgery, long travel, advanced age, and varicose veins. The primary medical treatment for DVT is the drug warfarin to prevent clots. Physical preventive measures are compression stockings and regular exercise of the calf muscle, at least every two hours, plus leg elevation.

For those at risk for DVT, regular exercise with a prescribed number of steps at least every two hours and periods of leg elevation can reduce the risk of clot development. Also the wearing of compression stockings can have a preventative effect.

Chronic wounds such as venous leg ulcers, diabetic foot or leg ulcers or pressure sores, which do not heal, represent a serious problem to sufferers and healthcare providers.

Various known treatments exist for the healing of such wounds. These treatments include applying compression bandaging to the wound area to help to reduce venous pressure and forcing venous return up the leg, elevating the lower leg to at least hip level and ideally with the foot above the level of the heart in order to aid blood flow return to the heart, and walking to exercise the calf muscles in order to improve the pumping action of the muscles to increase venous blood flow of the lower leg.

In the case of diabetic foot or leg ulcers, treatment consists of dressing the wound with a suitable compound and then placing a dressing over the wound followed by the application of what is known as an “off-loading boot” or cast to reduce pressure applied to the wound. Sufferers are required to wear the off-loading boot when putting weight on the foot.

Conventional treatment of pressure ulcers involves reducing the amount of time that external pressure is exerted on the wound area through lying or sitting, via the use of pressure reducing devices like specialized mattresses, beds and cushions and via frequent patient turning by the care provider. The wounds are dressed with suitable dressings.

An important factor in healing wounds is patient compliance with treatment regimes. Most patients suffering from wounds such as venous leg ulcers or diabetic foot ulcers are ambulatory and live in a community setting rather than in institutionalized care where treatment can be supervised by medical personnel. Consequently, treatment of the wound is greatly the responsibility of the patients themselves under the supervision of health care personnel who may visit once or twice weekly.

Unfortunately, since these patients are not monitored daily by health care personnel, non-compliance and treatment failures are very common.

For compression therapy to be effective in the treatment of venous leg ulcers, for example, a correctly applied compression bandaging should be left in place for a period of 5 to 7 days before changing. However, during a period of treatment, a patient will often unwrap a compression bandage and re-wrap it, either just before a Health Care Professional pays a visit or worse, shortly after the initial application. This is particularly likely to occur in warmer climates where the heat adds an extra degree of discomfort to wearing a heavy compression bandage.

In addition, from the perspective of health care personnel, applying compression bandaging correctly so as to achieve sustained compression for the duration of treatment requires a high degree of skill, particularly with multi-layer bandaging. Usually, a pressure of 30-40 mmHg at the ankle is required, gradually decreasing as you move up the leg to 15-25 mmHg below the knee. Achieving the required consistent gradients of pressures is challenging as not all medical personnel have achieved the required competencies, leading often to poorly applied bandages which are of limited efficacy as they do not apply a decreasing pressure gradient and that can easily loosen through no fault of the patient.

Moreover, despite the advice to walk around or elevate their leg to promote healing of a venous leg ulcer, many patients often spend much of their day sitting down, which is the least desirable possible position in terms of adding to the ‘pooling’ of blood in the lower leg. Venous leg ulcers can be very painful, with the pain invariably de-motivating the patient from moving around. The same rationale applies to the recommendation of keeping the leg elevated to promote healing, when not walking around.

While health care personnel may be motivated to help the patient, they are limited in their ability to influence the patient's behaviour outside the clinic. Health care personnel are totally reliant on the information provided by the patient at the weekly visit to understand if the patient has been compliant with advice. They have no way of knowing the extent to which patients are elevating their legs or walking around.

For example, when a patient arrives with a bandage which is loose, the Health Care Professional cannot know if this has been as a result of patient intervention or as a result of poor application. Similarly, if the patient arrives with the bandage unwrapped, the Health Care Professional cannot know when in the past treatment period the bandage was actually removed.

### SUMMARY OF INVENTION

A first aspect of the invention provides a patient compliance monitor for monitoring compliance of a patient to a treatment regime for treatment of a medical condition. The patient compliance monitor includes measurement means for measuring at least one external physical parameter acting on the body of the said patient, the at least one external physical parameter having an influence on the medical condition experienced by the body; recording means for recording over a period of time data representative of the physical parameter; and comparative means for comparing the recorded data with data indicative of the treatment regime to determine patient compliance to the treatment regime.

External physical parameters may comprise environmental factors, physical factors or ambient conditions experienced by the injured tissue. Examples of such physical parameters are ambient temperature, pressure applied to the limb, motion of the limb, tilt of the limb relative to gravity. Motion can include parameters representative of the physical activity of the patient such as the number of steps taken.

Medical conditions may include, for example, wounds, injured tissue such as burns, lymphedema, repetitive strain injury or any adverse medical condition resulting from a lack of activity or over-activity.

The patient compliance monitor allows data associated with a curative or preventative treatment regime to be provided and in so doing can positively influence a person's behaviour thereby increasing the probability of the treatment regime working. By encouraging preventative or pro-healing behaviour, and discouraging behaviour adverse to healing or prevention, a virtuous circle can be created. For example, if injured tissue that was previously not showing much healing progress is kick-started into a healing trajectory, a patient may experience both a reduction in pain and an increase in hope, both of which should motivate the patient to further comply with a Health Care Professional's advice.

A record of treatment can be created and checked by healthcare personnel to verify that the appropriate treatment is being maintained. Knowing that data representative of treatment is being recorded will encourage patients to comply with the treatment and thus help to promote preventative or curative behaviour. Moreover, healthcare personnel may be able to tailor treatment according to feedback and outcome of previous treatments. For example, healthcare personnel will be able to verify if compression bandaging has been removed from a limb or has become loose. They will also be able to detect when the compression bandaging has been removed or at what moment it became loose.

The record of data may also be used, for example, to assess insurance risks. For example, a patients medical insurance premium may be increased or decreased according to his or her compliance to a treatment regime.

The measurement means may include a tilt sensor for measuring the tilt of said limb relative to the direction of gravitational force. Thus, a patient may ensure that his or her leg is correctly positioned for effective prevention of conditions such as DVT or healing of wounds or injured tissue, when elevating the leg to aid blood flow to the heart.

The measurement means may include a movement sensor for monitoring motion of said limb, i.e. the physical activity of the person. Thus, data representative of patient movement may be collected and the patient and/or supervisor may verify that the patient is being active enough in flexing and/or strengthening the calf muscles to increase venous blood flow in the lower leg for the healing of wounds or injured tissue, or prevention of medical conditions such as DVT.

The measurement means includes a pressure sensor for measuring the pressure applied to a region of said limb.

In the case of curative treatment for venous leg ulcers or lymphedema, or the preventative treatment of DVTs, for example, it is thereby possible to see whether compression bandaging applied to a limb has been correctly applied and whether the correct application of pressure is being or has been maintained. In the case of treatment of diabetic foot ulcers, for example, it is thus possible to detect if excessive pressure is being applied to the wound area. In the case of the treatment of pressure sores, it is thus possible to detect if excessive pressure is being applied to the wound area. In the case of treatment of burns it is thus possible to verify that the correct pressure is being applied by a compression garment to the area of injured tissue for a sufficient period of time. In the field of orthotics the device allows one to verify if excess pressure is being applied to the skin where a prosthetic is fitted which may lead to the formation of skin breakdown and pressure ulcers.

The measurement means may include a thermometer for monitoring the ambient temperature around said limb. Thus, ambient temperature around said limb can be monitored. Ambient temperature can have an effect on wound healing. For example, if the patient is in cold surroundings cutaneous temperature may drop as a result of redirection of blood to the body core thus reducing blood flow available to assist healing. In hot ambient conditions there may be a higher risk of infection. The monitor may further comprises a thermometer for measuring cutaneous temperature. An increase in cutaneous temperature may indicate increased inflammation or infection, while a decrease in cutaneous temperature may indicate a loss of blood flow to the skin due to a systemic problem.

The movement sensor may comprise a pedometer operable to count the number of steps taken by said patient. In this way, the patient or health personnel analysing the data may verify that an appropriate number of steps for calf muscle exercise have been taken over a predetermined period of time such as on a daily basis.

The movement sensor may comprise means of measuring a period of time for which a patient is in motion.

The patient compliance monitor may have a display for displaying data associated with the physical parameter. In this way a patient can be provided with immediate feedback on his or her compliance to a treatment regime.

The display may be operable to indicate: the condition that a predetermined angle of tilt of the limb with respect to the direction of gravitational force has been reached or the period of time during which a predetermined angle of tilt has been applied. Thus, a patient can verify that his or her limb is being elevated at the correct angle or that it is been held at a correct angle for a sufficient period of time for the promotion of preventative or curative behaviour.

The display may be operable to indicate the number of steps taken by the patient, or the amount of time spent in motion, or the condition that a predetermined number of steps, or time in motion have been or have yet to be taken by the patient. Thus a patient can have immediate feedback as to whether he is being active enough for curative or preventative treatment.

The display may be operable to indicate the condition that a correct pressure, or gradient of pressure is being applied to the limb, that the pressure being applied to the region of said limb should be increased or reduced and or the absolute or average pressure being applied.

Thus, healthcare personnel may immediately see after applying compression bandaging, for the treatment of medical conditions such as venous leg ulcers, if compression bandaging has been applied properly. In addition, the wearer of the compression bandaging can be provided with immediate feedback on the condition of the compression bandaging and may see, for example, if the bandaging has become loose or if sufficient pressure is no longer being applied to the limb for preventative or curative treatment.

The display may be operable to be visible through a compression dressing. Thus, the display can be fitted on the limb of a patient and data can be viewed through the compression bandaging. In this way there is no need to have cables passing from one side of compression bandaging to the other.

The patient compliance monitor may include treatment delivery means for applying treatment to the body of the patient.

The treatment delivery means may be operable to apply therapeutic energy to the limb.

The treatment delivery means may be operable to apply one of electromagnetic, magnetic, heat, mechanical or ultra sonic energy to the body.

The treatment delivery means may comprise a plurality of electrodes for applying electrical signals to the region of said wound.

Thus, an additional form of treatment can be applied in conjunction with compression therapy, leg elevation and calf muscle exercise. For example, electrotherapy will help to stimulate a healing process or preventative process and also reduce pain related to the medical condition.

Electrical current can thus pass from one electrode to another electrode through different paths through the tissue under a wound or though injured tissue thereby providing more effective treatment, or permitting the progress of healing to be assessed.

Treatment may be delivered wirelessly to the region of said wound. Treatment may include electromagnetic, magnetic, heat, mechanical, ultrasound and interferential treatment.

The patient compliance monitor may include electrical generating means for generating electrical signals to be applied between electrodes of the plurality of electrodes. Thus an autonomous device may be provided.

The patient compliance monitor may include electrode connectors for supplying electrical signals to the electrodes wherein each of the electrode connectors has a first end adapted to pierce through a compression dressing for connection to an external electrical generator. This provides connection between electrodes and an external electrical generator to be facilitated. The connectors can also help to keep the bandage in place.

The patient compliance monitor may include a compression dressing arranged, in use, on said limb to reduce pooling of blood in the region of said wound. Thus, compression treatment may be applied in conjunction with monitoring. The patient compliance monitor may be fitted in the compression dressing.

The patient compliance monitor may include alert means for alerting the patient of non-compliance with the treatment regime.

Thus the patient can be immediately alerted that his behaviour is not complying with curative or preventative treatment or that a compression dressing has become loose, for example. In this way the patient can be reminded, for example to be more active in order to reduce the risk of an adverse health event such as the formation of DVT.

The alert means may be a vibrator to alert the patient by perceivable vibration or may be operable to emit an audible alarm or to generate a visible signal to alert the patient.

The patient compliance monitor may be disposed in a compressible housing, the housing being operable to compress under the influence of pressure exerted by a compression dressing such that it fits around at least part of the said limb.

This provides a comfortable fit when the device is being worn, especially when it is fitted under a compression dressing and helps to prevent additional pain being induced or the formation of sores. In this way it gives patients less temptation to remove the device because of discomfort.

The compressible housing can be made of a flexible elastomeric material or foam material. A side of the housing of the monitoring device may be contoured to fit around at least part of the limb.

The patient compliance monitor may be mounted on a limb mount, the limb mount being adapted to fit around at least part of the limb. The limb mount may be made of a flexible elastomeric material. A side wall of the limb mount may be contoured to fit against the limb of a patient.

The patient compliance monitor may include a detector for detecting removal of the patient compliance monitor from said limb. The detector may be operable to detect removal of the monitor according to the output of the measurement means. The detector may include a pressure sensor for detecting removal of pressure or may include a switch operable to open or close an electrical circuit when the patient compliance monitor is removed from the limb of the patient. This provides a sort of enforcement mechanism since an alert can be generated if the patient removes the monitoring device and/or a compression dressing. Thus the patient will be encouraged to wear the device thereby promoting curative and preventative treatment behaviour.

The patient compliance monitor may include data transmission means for transmitting recorded data to an external device so that the recorded data can be analysed by a physician.

In addition to measuring external physical parameters acting on said limb the temperature in a region of the wound, including ambient temperature, or the cutaneous skin or internal body temperature of the person or animal may be monitored. This allows temperature data to be collected which may help to anticipate breakout of bacteria and infection or to indicate the presence of inflammation.

A second aspect of the invention provides an ambulatory monitor for monitoring the risk of an onset or progression of a medical condition of a person. The ambulatory monitor includes measurement means for measuring an external physical parameter acting on a limb of said person, the external physical parameter having an influence on the onset or progression of a medical condition; recording means for recording, over a period of time, data representative of the physical parameter; comparative means for comparing the recorded data with data indicative of the onset or progression of the medical condition and alert means for generating an alert if the comparison performed by the comparative means indicates a risk of an onset or progression of a medical condition, wherein the measurement means comprises at least one of motion monitoring, means for monitoring motion of said limb and tilt monitoring means for monitoring the tilt of said limb relative to the direction of gravitational force.

Such a device can be used to alert a user that due to under-activity or over-activity he is increasing the risk of the onset of a medical condition or the worsening of a pre-existing medical condition. The device can thereby remind him to move or elevate his limb in order to reduce the risk of an adverse medical event such as the formation of DVT or warn him that he is at risk of a repetitive strain injury due to over activity. Motion can include physical parameters representative of the physical activity of the user such as number of steps taken.

A third aspect of the invention provides a monitoring device for monitoring treatment of a wound on a limb of a person or an animal, the device comprising monitoring means for monitoring external physical parameters acting on the said limb, said physical parameters having an influence on the healing of a wound on the said limb; and treatment delivery means for applying treatment for wound healing to the region of said wound.

With such a device, an additional form of treatment can be applied in conjunction with monitoring relating to compression therapy, leg elevation and/or calf muscle exercise. The treatment can help to stimulate a healing process or preventative process and also reduce pain related to a medical condition.

The treatment delivery means may be operable to apply a therapeutic form of energy to the region of said wound.

The treatment delivery means may be operable to apply one of electromagnetic, magnetic, heat, mechanical or ultra sonic energy to the limb.

The treatment delivery means may comprise a plurality of electrodes for applying electrical signals to the region of said wound.

The aforesaid electrodes may also or alternatively be used to monitor changes in the electrical properties of the wound area.

A fourth aspect of the invention provides a patient compliance monitor attachable to a patient for monitoring treatment of a medical condition experienced by a patient. The device includes monitoring means for monitoring physical factors acting on a said patient, said physical factors having an influence on the medical condition; and a detector for detecting detachment of the compliance monitor from said patient.

Physical factors may include presence of an object such as a medical treatment appliance on the patient, as well as external physical parameters including pressure, tilt, motion etc.

The device according to this aspect of the invention provides a sort of enforcement mechanism since an alert can be generated if a patient removes the monitoring device and/or a compression dressing. Healthcare personnel can be informed by an immediate alert or from analysis of data representative of the detector status if the monitoring device and/or a compression dressing have been removed, even if subsequently replaced. Thus a patient will be encouraged to wear the device and/or compression dressing thereby promoting curative and preventative treatment behaviour. The detector may be operable to detect detachment of the compliance monitor according to the output of the monitoring means. For example, a pressure sensor may be used to detect pressure applied to a treatment region as well as the presence of a medical appliance such as a compression dressing on the treatment region.

The monitoring means may comprise a pressure sensor for detecting removal of pressure.

The monitoring means may comprise a switch operable to open or close an electrical circuit when the patient compliance monitor is fitted against the patient.

The monitoring means may comprise a light sensor arrangement operable to detect the presence or absence of light.

A fifth aspect of the invention provides a monitoring device for monitoring treatment of a wound on the limb of a person. The device comprises monitoring means for monitoring external physical parameters acting on said limb, said physical parameters having an influence on the healing of a wound on said limb; and a compressible support arranged to compress to fit against a part of the limb under the influence of a force applied by a compression dressing.

Such a device provides a comfortable fit when worn, especially when it is fitted under a compression dressing and thereby helps to prevent additional pain being induced or the formation of sores. In this way it gives patients less temptation to remove the monitoring device because of discomfort.

The compressible housing may be a housing in which the monitoring means is disposed to a leg mount on which the monitoring means is disposed.

A sixth aspect of the invention comprises a method of monitoring the risk of an onset or progression of a medical condition of a person. The method comprises measuring at least one external physical parameter acting on a limb of said person, the at least one external physical parameter having an influence on the onset or progression of a medical condition; recording, over a period of time, data representative of the at least one physical parameter; comparing the recorded data with data indicative of the onset or progression of the medical condition and generating an alert if the comparison indicates a risk of an onset or progression of a medical condition, wherein the measuring an external physical parameter comprises at least one of monitoring motion of said limb and monitoring the tilt of said limb relative to the direction of gravitational force.

A seventh aspect of the invention provides a method of monitoring compliance of a patient to a treatment regime for treatment of a medical condition. The method includes measuring at least one external physical parameter acting on the body of the said patient, the external physical parameter having an influence on the medical condition experienced by said body; recording over a period of time data representative of the at least one physical parameter; and comparing the recorded data with data indicative of the treatment regime to determine patient compliance to the treatment regime.

An eighth aspect of the invention provides a method of monitoring treatment of a wound on the body of a person or an animal. The method comprises monitoring at least one external physical parameter acting on the said body, said physical parameters having an influence on the healing of a wound on the said body; and applying treatment for wound healing to the region of said wound.

A ninth aspect of the invention provides a method of monitoring compliance of a patient to a treatment regime for treatment of a medical condition, the method comprising monitoring physical factors acting on said patient, said physical factors having an influence on the medical condition; and detecting detachment of the compliance monitor from said patient.

A tenth aspect of the invention provides a portable treatment monitor for monitoring treatment of a wound on the body of a person, the treatment monitor comprising a monitor for monitoring at least one external physical parameter acting on the said body, said at least one physical parameter having an influence on the healing of said wound; and a treatment delivery appliance for applying treatment for wound healing.

Embodiments of the invention will now be described, by way of example only, and with reference to the following drawings in which:—

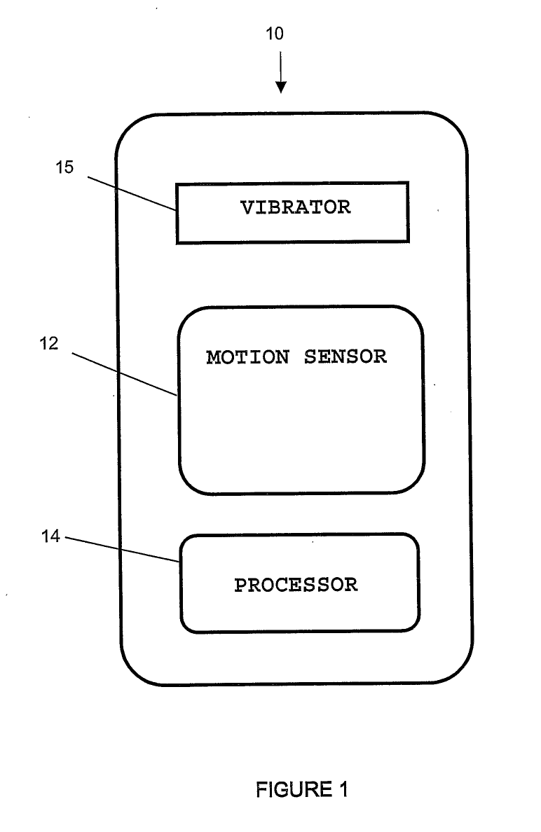

FIG. 1 is a schematic diagram of a first embodiment of an ambulatory monitor according to the invention;

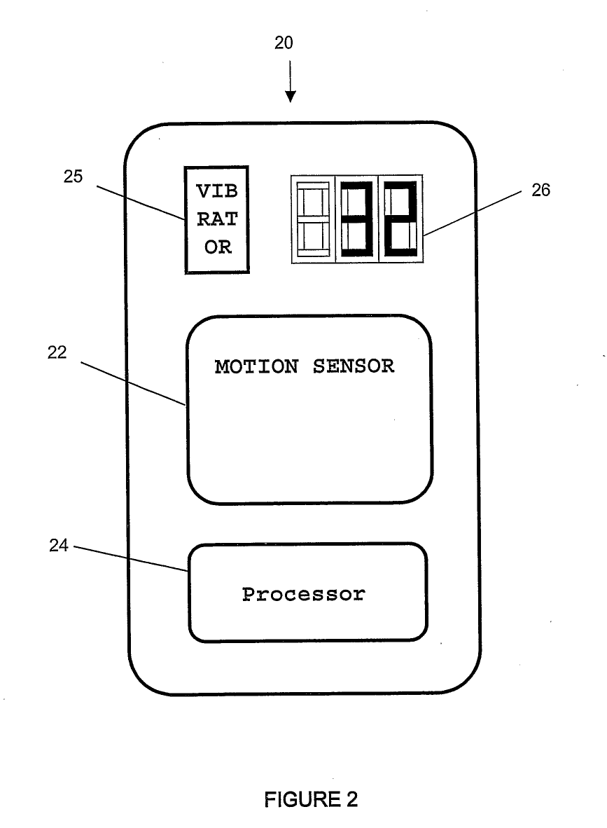

FIG. 2 is a schematic diagram of a second embodiment of an ambulatory monitor according to the invention;

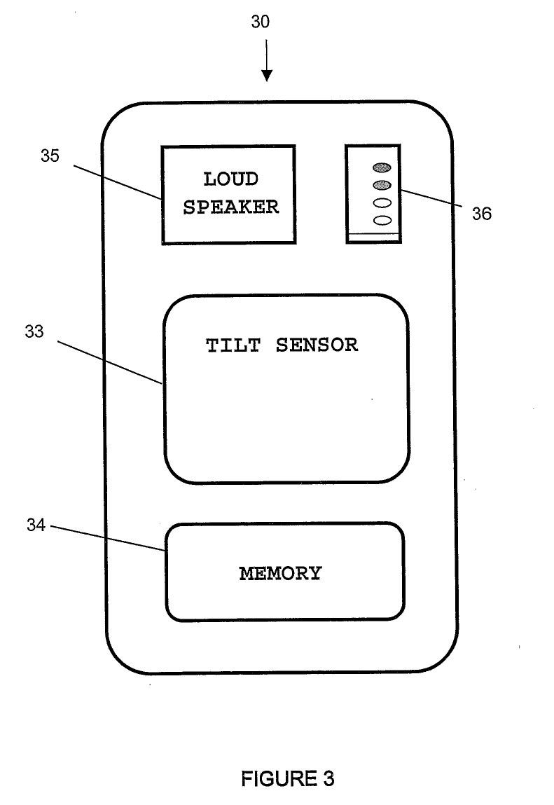

FIG. 3 is a schematic diagram of a third embodiment of an ambulatory monitor according to the invention;

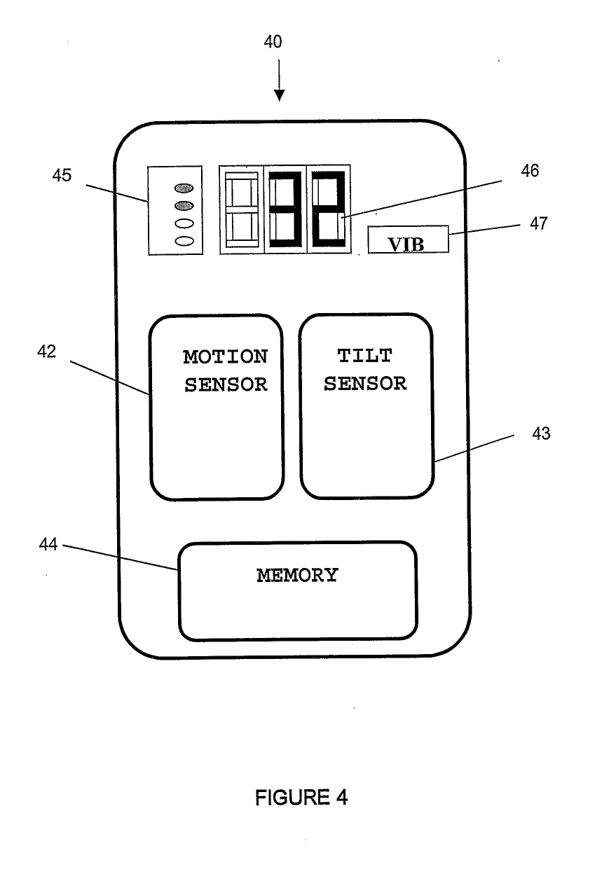

FIG. 4 is a schematic diagram of a fourth embodiment of an ambulatory monitor according to the invention;

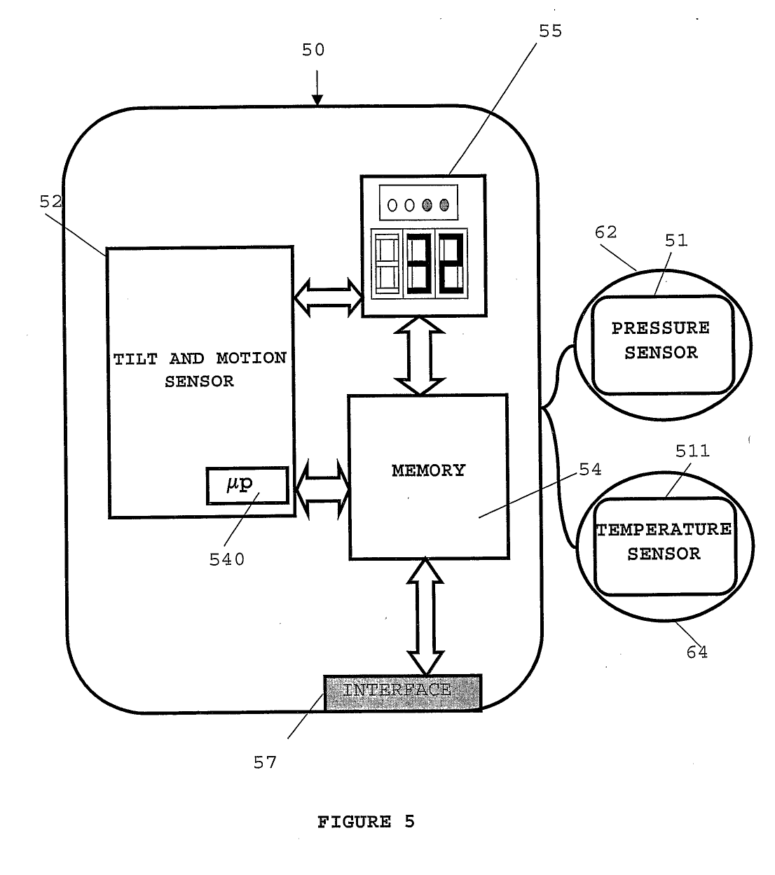

FIG. 5 is a schematic diagram of a fifth embodiment of an ambulatory monitor according to the invention;

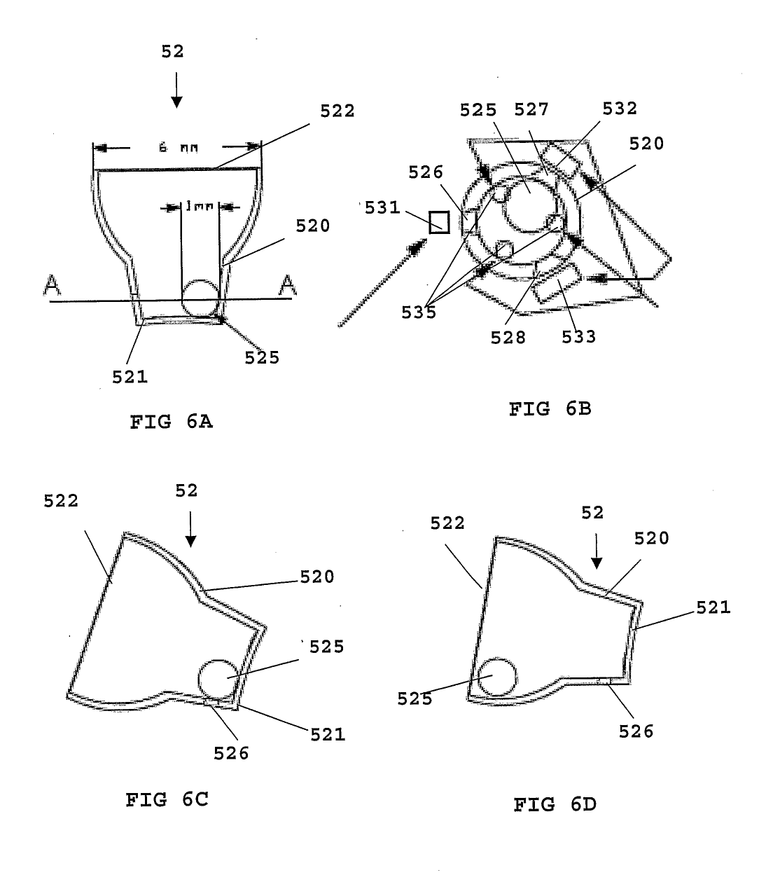

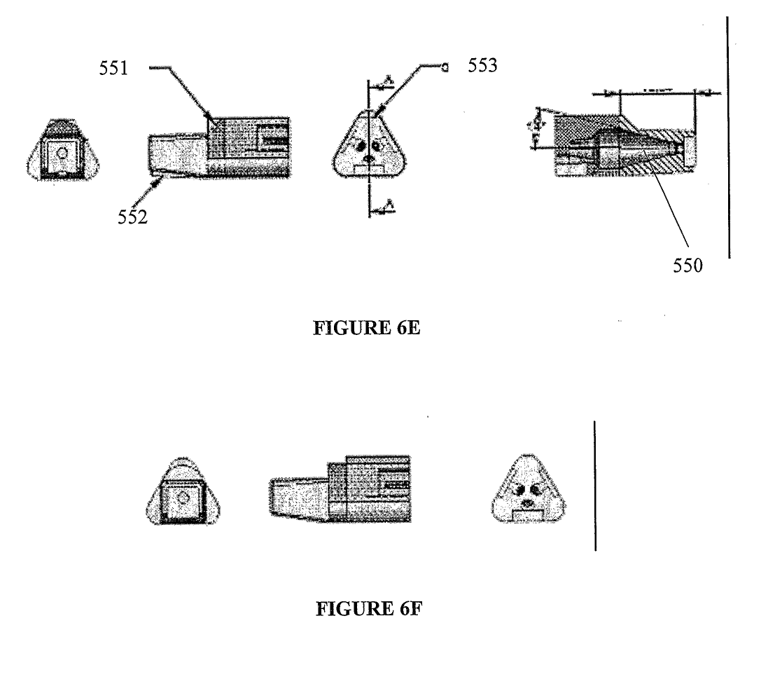

FIG. 6A is a schematic diagram of a tilt and motion sensor according to the fifth embodiment of the invention;

FIG. 6B is a cross sectional diagram of FIG. 6A taken along line A-A;

FIG. 6C is a schematic diagram of a tilt and motion sensor according to the fifth embodiment of the invention in a first position of tilt;

FIG. 6D is a schematic diagram of a tilt and motion sensor according to the fifth embodiment of the invention in a second position of tilt;

FIG. 6E illustrates an alternative embodiment of part of a tilt and motion sensor;

FIG. 6F is a cross sectional diagram of an embodiment of part of a tilt and motion sensor;

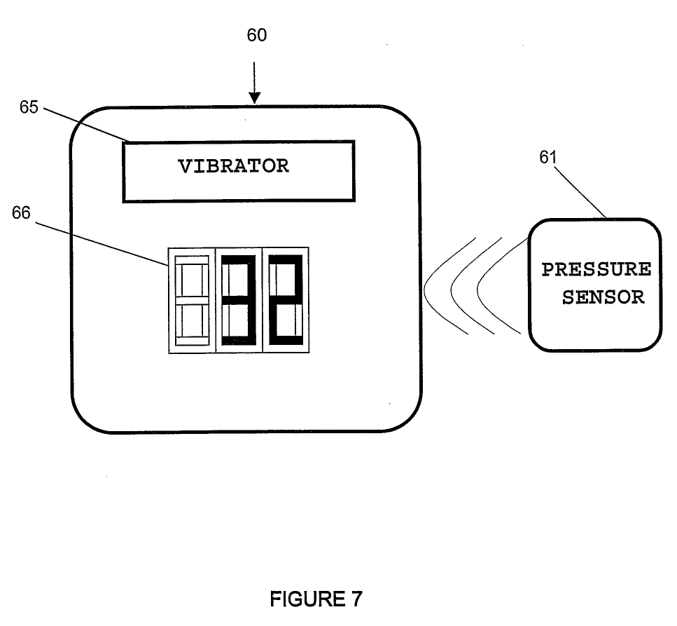

FIG. 7 is a schematic diagram of a patient compliance monitor according to a sixth embodiment of the invention;

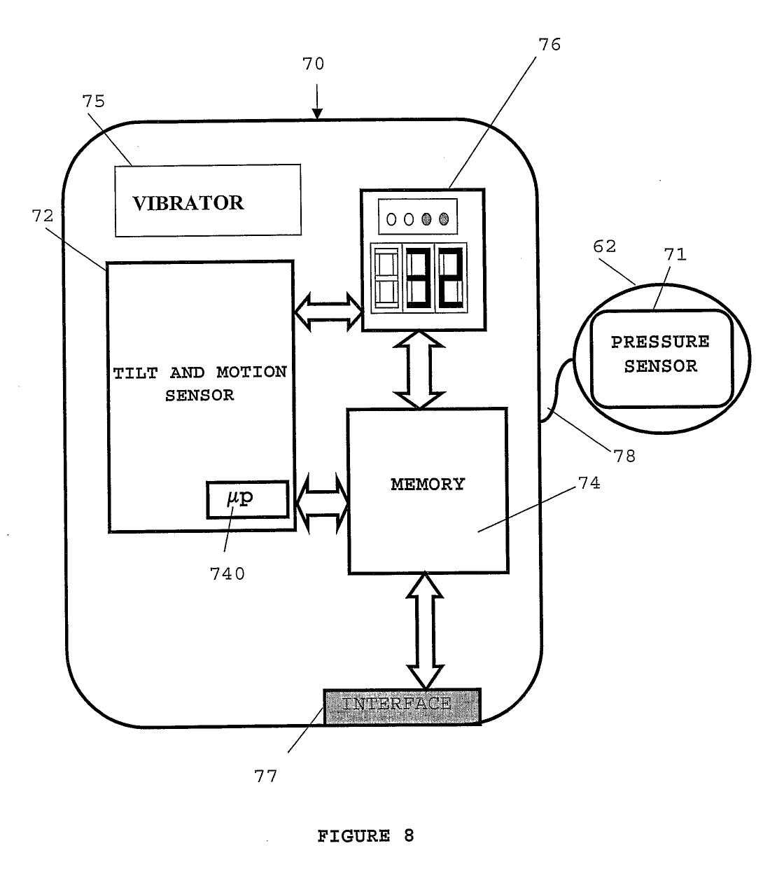

FIG. 8 is a schematic diagram of a patient compliance monitor according to a seventh embodiment of the invention;

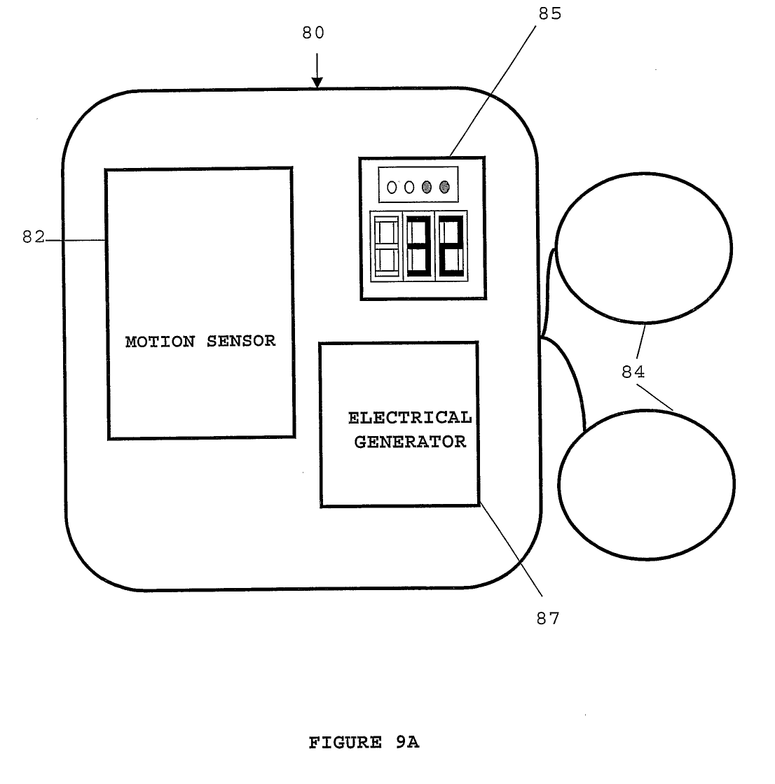

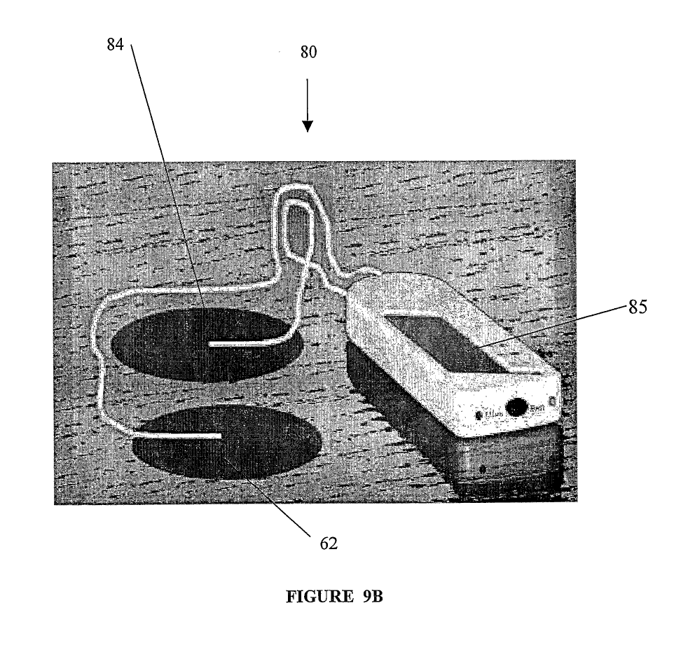

FIG. 9A is a schematic diagram of a patient compliance monitor according to an eighth embodiment of the invention;

FIG. 9B is a perspective view of patient compliance monitor of FIG. 9A;

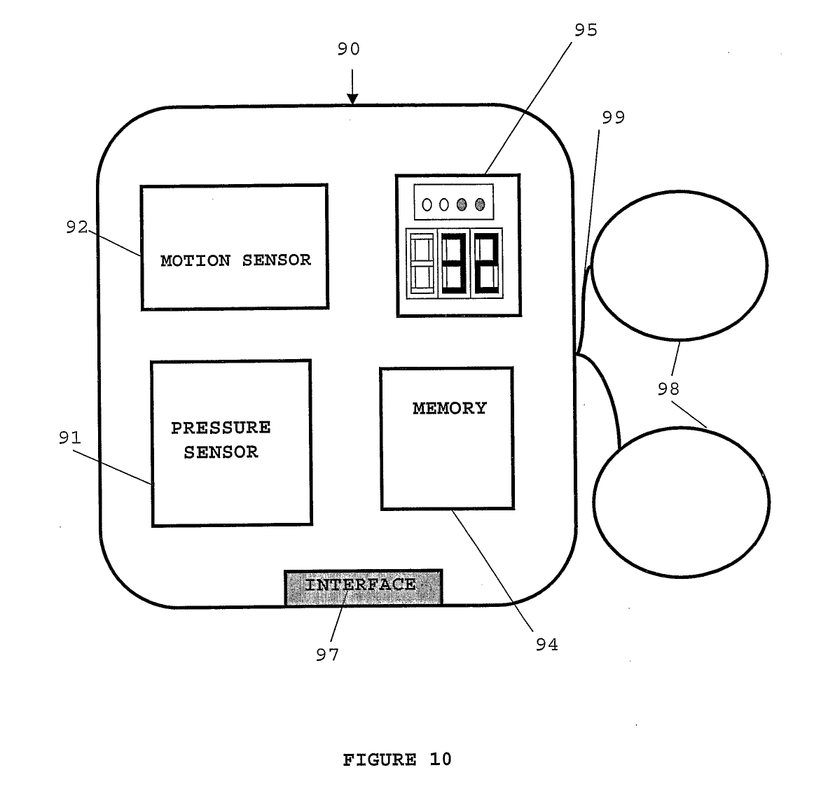

FIG. 10 is a schematic diagram of a monitoring according to a ninth embodiment of the invention;

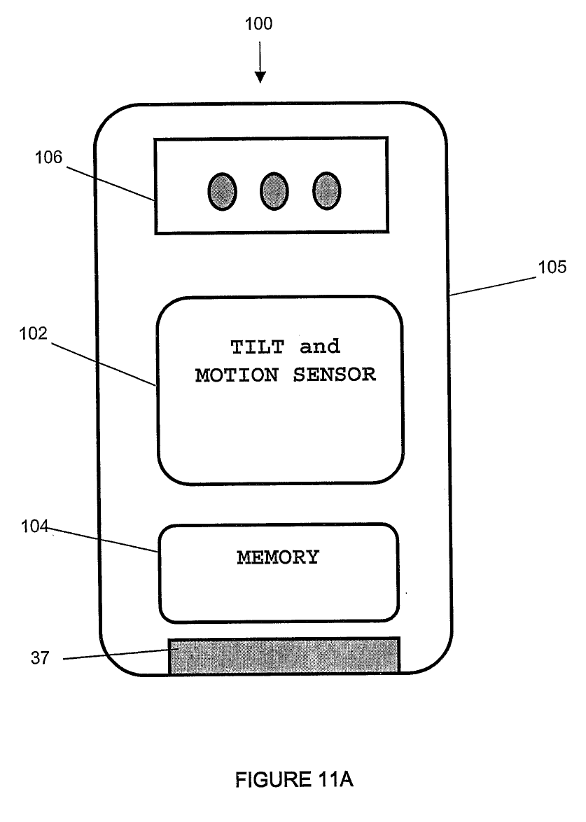

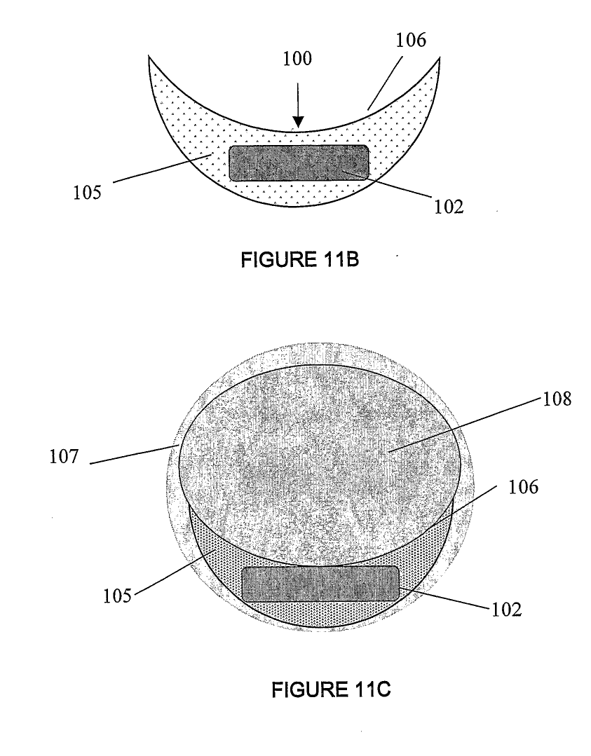

FIG. 11A is a schematic diagram of a monitoring according to a tenth embodiment of the invention;

FIG. 11B is a sectional view of the housing of the patient compliance monitor of FIG. 11A;

FIG. 11C is a sectional view of the housing of FIG. 11B fitted on a limb and wrapped in a compression dressing;

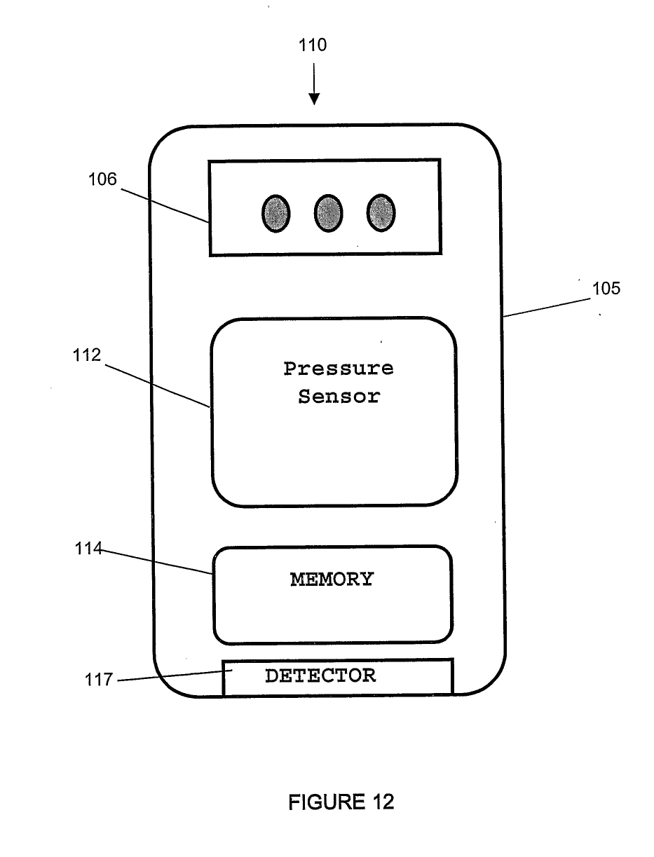

FIG. 12 is a schematic diagram of a monitoring device according to an eleventh embodiment of the invention;

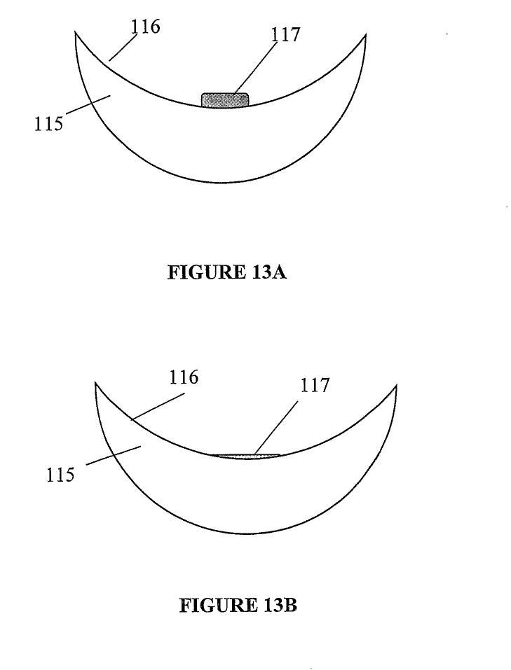

FIG. 13A is a sectional view of the housing of the patient compliance monitor of FIG. 12 illustrating the configuration of the removal detector when the device is not fitted on a limb; and

FIG. 13B is a sectional view of the housing of the patient compliance monitor of FIG. 12 illustrating the configuration of the removal detector when the device is fitted on a limb; and

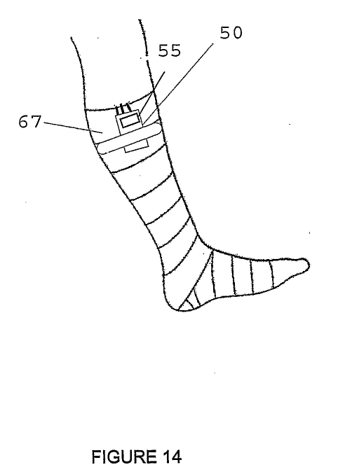

FIG. 14 is a schematic diagram of an embodiment of a method of monitoring treatment of a wound according to the invention.

### DETAILED DESCRIPTION

Referring to FIG. 1 a first embodiment of an ambulatory monitor 10 according to the invention comprises a motion sensor 12, a processor 14 and a vibrator 15. In this embodiment the motion sensor 12 is a pedometer and is configured to count the number of steps taken by a wearer. Such devices are well known in the art. The processor includes a timing device for defining a time period for counting the number of steps taken and a reset mechanism for resetting the number of steps to zero. The time period is adjustable and may, for example, be of a period of a few hours. The ambulatory monitor can be fitted on the limb of a user. If a predetermined number of steps is not taken during the predetermined time period the vibrator generates a vibration to alert the wearer and remind him or her to move in order to prevent the onset or worsening of a medical condition such as DVT.

Such a device would find use, for example, in the prevention of DVT during air flights where a person can be at an increased risk of a DVT due to the prolonged period of sitting down. The device would remind the wearer by means of a gentle vibration to get up and walk around the aircraft or to carry out recommended foot and leg exercises at his or her seat.

A second embodiment of an ambulatory monitor 20 is illustrated in FIG. 2. The ambulatory monitor is similar to that of the first embodiment and further includes a seven segment type display 26. Display 26 is configured to indicate the number of steps taken by a user. In alternative embodiments of the invention, the display 26 may indicate the remaining number of steps to be taken in order to achieve a predetermined target. Thus the wearer has the convenience of seeing the level of activity he has undertaken or what further activity he must undertake in order to comply with a curative or a preventative treatment regime, or to avoid the onset or worsening of a medical condition. The step count displayed on the screen can be manually reset to zero by the user or can be automatically reset to zero by the processor after a certain time period.

The devices of the first and second embodiments help to give the wearer the incentive to walk around and to achieve the recommended number of steps to be taken for a predetermined time period, for example per hour or per day for the prevention of the onset or progression of a medical condition. In the case of prevention of a DVT, for example, regular exercise of the calf muscle by walking helps to prevent the formation of a blood clot. A recommended number of steps or leg movement may be given for this prevention.

In the case of the treatment of venous leg ulcers, for example, improving calf muscle strength by walking helps to improve the pumping action of the muscles, thereby increasing the venous blood flow of the lower leg. Typically, a Health Care Professional recommends and instructs a patient on how much they should walk based on their physical condition. Thus a target number of steps can be set according to the recommended treatment.

In a further embodiment, the ambulatory monitor may further include a memory for recording the number of steps taken by a wearer during one or more predetermined time periods, or for recording the number of steps versus time over a period of time.

While in these embodiments the motion sensor is located in the same housing as the display and processor, in alternative embodiments of the invention the motion sensor may be remote to the display and memory and communicate with the display and processor via a cable or wireless link.

Referring to FIG. 3 a third embodiment of an ambulatory monitor comprises a tilt sensor 33, a memory 34, an audible alarm 35 and a tilt sensor display 36. The tilt sensor 33 is an inclinometer configured to measure the angle of the limb of a person relative to the direction of gravitational force i.e. relative to a line of gravity, when the monitor is fitted on the limb of the user. Such devices are well known in the art and include devices based on the movement of conductive fluid, a pendulum or contacting elements. Measurements of the tilt angle are recorded in memory 34 of the device 30. Tilt display 36 is configured to indicate elevation when an acceptable angle of tilt has been achieved.

In an alternative embodiment of the invention, the tilt display 35 may be configured to indicate when the limb of a patient has been held at a recommended angle of tilt for a recommended time period.

Audible alarm 37 is a loudspeaker configured to emit an audible alert when the user has been inactive, i.e. has not held his or her leg at the required angle for a sufficient period of time.

An elevation may be typically defined as around 15 degrees above the horizontal, the horizontal being perpendicular to the direction of gravitational force. In the case of monitoring the angle of leg tilt for the treatment of venous leg ulcers the recommended angle of tilt would be in the range of from 0 degrees to the horizontal to 30 degrees above the horizontal.

The indication of tilt helps to encourage the patient to keep the leg elevated when he or she is not mobile. Elevating the lower leg above the hip level aids blood flow return to the heart. Ideal elevation comprises bringing the foot above the level of the heart as this action helps to drain the lower leg with the aid of gravity while bringing the leg to a horizontal position helps to reduce venous pressure build-up or the formation of blood clots.

Despite the advice to walk around or elevate their leg, many people often spend much of their day sitting down, which is the least desirable position since it encourages ‘pooling’ of blood in the lower leg. The audible alarm helps to remind a person to elevate his or her leg.

It will be appreciated that in any of the embodiments the alarm may use any audible, visual or vibration signal either alone or in combination.

In an alternative embodiment, two sensors, of any suitable technology, capable of assessing each other's relative position in the vertical axis, one worn on the lower limb, preferably on or near the foot with the ulcer, and the other worn on or near the torso, preferably in proximity of the patient's heart, will permit a calculation of whether the foot has been raised above the heart level and drive both the indicator and non-compliance alert accordingly.

Referring to FIG. 4 a fourth embodiment of an ambulatory monitor comprises a motion sensor 42, a tilt sensor 43, a memory 44, a tilt display 45, a step display 46 and a vibrator alarm 47. Motion sensor 42 is substantially identical to motion sensor 12 of the first embodiment and tilt sensor 43 is substantially identical to tilt sensor 33 of the third embodiment. Measurements of the tilt angle and the number of steps taken during set time periods are recorded in memory 44 of the device 40. Tilt display 45 is configured to indicate when an acceptable angle of tilt has been achieved. Step display 46 is configured to display the number of steps taken by the user. In an alternative embodiment of the invention, the tilt display 45 is configured to indicate when the limb of a patient has been held at a recommended angle of tilt for a recommended time period. In a further embodiment of the invention the step display 46 is configured to display the number of steps remaining in order to achieve a predetermined target.

Vibrator 47 is configured to generate a perceivable vibration when the user has been inactive for too long, i.e. has not walked the required number of steps and/or has not held his or her leg at the required angle for a sufficient period of time.

Although in this embodiment a vibrator is used to warn the user of inactivity, in alternative embodiments of the invention the alert means may be an audible alarm or a visual display emitting a visible warning signal such as a red flashing light.

Referring to FIG. 5, a fifth embodiment of an ambulatory monitor 50 according to the invention comprises a pressure sensor 51, a temperature sensor 511, an integrated motion sensor and tilt sensor 52, a memory 54, a display 55 and a data interface 57.

Pressure sensor 51 is configured to measure the pressure applied to soft tissue on the limb of a user. Temperature sensor 511 is configured to record the cutaneous temperature of the user. The pressure sensor 51 and the temperature sensor 511 are located in pads 62 and 64, respectively remote to the main unit 50 housing the motion and tilt sensor 52.

Readings of pressure, temperature, number of steps taken, tilt angle over time are recorded in memory 54.

Display 55 is configured to indicate that the current pressure and/or temperature reading are within a predetermined range, the number of steps that have been taken and when an acceptable angle of tilt has been achieved according to a preventative or curative treatment regime.

In alternative embodiments of the invention the display 55 may indicate the remaining number of steps to be taken in order to achieve a predetermined target. In a further embodiment of the invention, the display 55 may configured to indicate when the limb of a patient has been held at a recommended angle of tilt for a recommended time period. In an even further embodiment of the invention the display may indicate the pressure reading and/or the temperature reading.

It will be appreciated that while the ambulatory monitor of this embodiment has one display unit common to all the sensors, in alternative embodiments of the invention each sensor may have its own dedicated display.

While in this embodiment the pressure sensor and temperature sensor are connected to the main unit by means of wires, in further embodiments of the invention the temperature sensor and/or the pressure sensor may be linked to the main unit by a wireless link. In even further embodiments of the invention the pressure sensor and the temperature sensor may be located in the same unit.

It will also be appreciated that while in this embodiment the tilt sensor and motion sensor are combined in a single unit, in alternative embodiments the ambulatory monitor may include a separate motion sensor and tilt sensor such as those of the fourth embodiment.

After a session, data recorded in memory 54 can be downloaded to a computer or PDA via an interface 57 for analysis. The interface 57 may be directly connected to a corresponding computer for data downloading. In alternative embodiments of the invention the data may be transferred from the device to a computer using a wireless connection. Moreover, in further embodiments data may be transmitted by GPS/WAP or by telephone for data telemedicine.

The integrated tilt and motion sensor 52 will be described in more detail with reference to FIGS. 6A to 6D. An integrated tilt and motion sensor according to this embodiment of the invention comprises housing 520 and a ball bearing 525 which is free to move within the housing 520. The housing 520 is of a cylindrical shape and tapers from a first end 521 towards a second opposite end 522. The diameter of the housing at the first end 521 is approximately twice the diameter of the ball bearing 525. The height of the sensor housing 520 is approximately 20 mm, and the diameter of the sensor housing 520 ranges from approximately 2 mm at the first end 521 to approximately 6 mm at the second end 522.

Three slots 526, 527, 528 are located at positions of 120° intervals around the circumferential wall of the housing 520. An infra red emitter 531 is positioned outside the housing 520 and facing towards the slot 526. Infrared sensors 532 and 533 are positioned outside the housing 520 and facing towards slots 527 and 528, respectively. Protrusions 535 located on the inner wall of the first end 521 determine the sensitivity of the sensor 52 and ensure that when the tilt and motion sensor is at rest the ball bearing 525 does not move.

In use, the ambulatory monitor 50 is strapped to the lower leg so that the longitudinal axis of the cylinder housing 520 extends parallel to the direction of gravitational force when the user is in a standing position.

When the leg to which the monitoring device 50 is attached, is moved the ball bearing 525 will move to obstruct at least one of the slots 526, 527 or 528 thereby breaking the path of infrared light between the infra red emitter 531 and one or both of the infrared sensors 532 and 533.

The signals received from infrared sensors 532 and 533 are processed by a processor 540 to provide a motion pattern induced by the ball bearing as it moves in the housing 520. The processor 540 is configured to determine the number of steps taken by the patient from the motion pattern

When the leg is elevated the ball bearing 525 will move to obstruct at least one of the slots 526, 527 or 528 thereby breaking the path of infrared light between the infra red emitter 531 and one or both of the infrared sensors 532 and 533.

The cylinder housing 520 is tapered so that as the leg is further elevated, the ball bearing 525 rolls from the first end 521 to the second end 522 of the housing 520. In this way each of the apertures 531, 532 and 533 are open allowing infrared light from infrared emitter 531 to reach both infrared sensors 532 and 533. The processor 540 can thereby detect the absence of ball bearing 525 from the signals received from infrared sensors 532 and 533 at the first end 521 of the housing 520 and deduce that the leg is elevated.

It will be appreciated that by suitable shaping of the housing and the addition of further infrared emitters and/or infrared sensors it is possible to distinguish different angles of elevation of the leg. In particular, if the flat end of the housing (uppermost in FIG. 6A) is replaced by a cone tapering 550 to a new emitter at the top, and emitter 531 is replaced with a further sensor, it is possible to distinguish a particular minimum angle of elevation where the ball bearing 525 rolls to the tip of the cone and interrupts the path of infrared light from the new emitter to all the sensors from the horizontal condition where the ball will be as in FIG. 6D and all sensors will be illuminated. This is illustrated in FIG. 6E in which the housing has an added chamfer 551 and an added rib 552 with respect to the previous embodiment shown in FIG. 6F.

The processor 540 is configured such that either the tilt or motion function may be disabled so that the sensor can function solely as a motion sensor or solely as a tilt sensor.

A patient compliance monitor 60 according to a sixth embodiment of the invention is illustrated in FIG. 7 and comprises a pressure sensor 61, a vibrator 65 and a display unit 66. The pressure sensor 61 is configured to communicate with the display unit 66 and the vibrator 65 by means of inductive wireless communication.

The pressure sensor 61 may be a semiconductor, resistive or any proprietary small pressure measuring transducer.

For example, the pressure sensor may be a pressure sensor made of Quantum tunneling composite (QTC), a pressure sensor made up of a series of membrane switches that are designed to operate at different pressures by varying the material of the membrane or aperture in the spacer between the top and bottom contacts, a pneumatic sensor using a sealed partially inflated “sausage shaped balloon” to which is attached a single pressure sensor to take the average pressure of a compression bandage over that length or any other suitable pressure transducer known in the art.

In order to ensure that the pressure sensor 61 does not record pressure at a single point the pressure sensor 61 is mounted on a substrate to spread the loading pressure over as large an area as possible. In this way the pressure sensor can average the pressure over a significant area.

In alternative embodiments of the invention the pressure sensor 61 may be made up of multiple small pressure sensors spread over an area of treatment. This would enable the measurement of a whether a suitable relative pressure gradient is achieved or not. The multiple pressure sensors may be provided on one strip or alternatively they may be separate to one other and configured to communicate wirelessly to provide a pressure gradient.

Vibrator 65 is configured to vibrate when insufficient or excessive pressure, from, for example a compression dressing or a burns pressure garment is being applied to the area of treatment.

Display 66 includes a seven-segment display and is configured to indicate the pressure measured by the pressure sensor 66. In alternative embodiments the display is configured to indicate that the measured pressure falls within a predetermined pressure range. In the case of monitoring compression therapy for venous leg ulcers, for example, the predetermined range of pressure applied by compression bandaging to the area of treatment should be of from approximately 30 to 40 mmHg at the ankle reducing gradually to 15 to 25 mmHg below the knee. The display unit emits visible light which can pass through one or two layers or standard compression bandaging so that the reading on the display is visible through the compression dressing.

In an alternative embodiment of the invention instead of having a display visible through compression dressing a display unit may be placed on the outside of the compression bandaging and may be in inductive wireless communication with the pressure sensor 61. In this way the display is visible and there are no wires which may impede the process of compression bandaging or which may press into the leg of the patient possibly leading to the development of further sores on the fragile skin of venous leg ulcer patients.

In use, for the treatment of venous leg ulcers, the pressure sensor 61 can be placed in contact with soft tissue in the region of an ulcer under compression bandaging.

Indicating that the correct pressure is being applied to the wound area allows medical personnel applying a compression bandaging to ensure that the compression bandaging has been applied correctly. As mentioned above, applying compression bandaging correctly so as to achieve sustained compression for the duration of the treatment requires a high degree of skill, particularly with multi-layer bandaging. Achieving the required consistent gradients of pressures can be difficult and can lead to poorly applied bandages that can easily loosen thereby becoming ineffective. Furthermore, the patient can check that the correct pressure is being maintained throughout the duration of the treatment.

A patient compliance monitor 70 according to a seventh embodiment of the invention is illustrated in FIG. 8 and comprises a pressure sensor 71, a tilt and motion sensor 72, a vibrator 75 and a display unit 76. The pressure sensor 71 is remote to the monitor and is similar to the pressure sensor of the sixth embodiment but is configured to communicate with the monitor by means of a cable 78. Cable 78 is made of fibre woven cable for supplying signals to the display unit. The fabric cable helps to prevent the skin of the user from being broken and the development of sores since it reduces localised pressure against the leg of the user.

Readings of pressure, movement data, tilt angle over time are recorded in memory 74.

Display 76 is configured to indicate that the current pressure and/or temperature reading are within a predetermined range, when an acceptable amount of activity has been taken and when an acceptable angle of tilt has been achieved according to a treatment regime.

The vibrator 75 is configured to generate a perceivable vibration when the patient has been inactive for a period of time, has not held his leg at an angle for a long enough period of time, and/or when the pressure applied does not fall with a recommended pressure range. The display 76 indicates which part of the treatment regime has not been complied with.

In alternative embodiments of the invention different forms of data representative of the measured physical parameters may be displayed.

After a treatment session, data recorded in memory 74 can be downloaded to a computer or PDA via an interface 77 for analysis by a physician or health care personnel. The interface 77 may be directly connected to a corresponding computer for data downloading. In alternative embodiments of the invention the data may be transferred from the device to a computer using a wireless connection.

In an alternative embodiment data may be transmitted by GPS/WAP or by telephone for data telemedicine.

Such a device allows a patient to see whether or not he is complying with a treatment regime and alerts the patient when the treatment regime has not been complied with. Further a physician or health care personnel can be provided with data recorded during a treatment period and may determine if a particular regime has been complied with by comparing the data with data indicative of the treatment regime.

The recording of pressure data over time allows medical personnel to see if compression therapy has been correctly applied over the duration of treatment. The pressure data taken over the duration of treatment helps to identify if the patient has removed the compression bandaging or if the compression bandaging has become loose and thus has not applied sufficient pressure to the wound area over the course of treatment.

If, for example, a patient arrives with a bandage that is loose, the Health Care Professional will be able to find out if this has been as a result of patient intervention or as a result of poor application. Similarly if the patient arrives with the bandage unwrapped, the Health Care Professional will be able to find out when, during the preceding treatment session, the bandage was actually removed.

From the point of view of the patient, knowing that the pressure is being monitored over the course of treatment will discourage him or her from removing the compression bandaging and thus will promote healing of the wound.

It will be appreciated that in alternative embodiments of the invention, the ranges of pressure indicating effective treatment will vary according to the treatment required. It will also be appreciated that the duration of treatment monitoring and the frequency of pressure readings may be adapted to the treatment involved.

For example, in a further embodiment of the invention the device may be used to monitor the treatment of pressure sores and may be configured to alert medical personnel that pressure applied to a particular area of tissue has exceeded a predetermined level.

In alternative embodiments of the invention, the device may be used to monitor the treatment of diabetic foot ulcers and to alert the patient or medical personnel if excessive pressure is being applied to the area of the ulcer. Referring to FIGS. 9A and 9B, a monitoring device according to an eighth embodiment of the invention comprises a motion sensor 82, a display 85, a pair of electrodes 84 for applying electrical signals to the skin of a patient when placed on the skin of a patient and an electrical generator 87 for supplying the electrodes 84 with the electrical signals. Motion sensor 82 is similar to motion sensor 12 of the first embodiment. Display 85 is arranged to indicate the number of steps taken by the user and to emit a warning visual signal if the required number of steps have not been taken within a set time period.

In use, in for example, the treatment of wounds, the electrodes can be placed in a peripheral area of the wound. Current passing from one electrode to the other will thereby pass through regenerative tissue under the wound promoting healing of the wound. This allows electrotherapy to be included in a treatment regime, thus helping to promote healing of the wound in addition to physical activity of the patient monitored by the motion sensor.

Although in this embodiment the treatment monitor is provided with a pair of electrodes, it will be appreciated that in alternative embodiments of the invention, the treatment monitor may be provided with three or more electrodes and the electrical generator may be configured to apply current between any electrodes of the set of electrodes so that current passes through different paths in the regenerative tissue.

Referring to FIG. 10 a portable treatment monitor according to a ninth embodiment of the invention comprises a pressure sensor 91, a motion sensor 92, a memory 94 for recording data from the pressure sensor and the motion sensor, a pair of electrodes 98 for applying electrical signals to the skin of a patient, electrode connectors 99 for relaying electrical signals to the electrodes 98, a display 95 and a data interface 97 through which data can be downloaded from the memory 94.

Display 95 can indicate the number of steps taken by the user or the number of remaining steps to be taken by a user in order to hit a target number of steps and indicates if a correct pressure is being applied to an area of treatment.

In further embodiments the portable treatment monitor may include alert means such as a vibrator, audible alarm or visual alarm to warn the patient that an insufficient number of steps have been taken or that an incorrect pressure is being applied.

In use, for treatment of a wound under a compression dressing the electrodes are placed in the peripheral area of the wound under a compression dressing and an external electrical generator is placed outside the compression dressing. Electrode connectors 99 each have a pierced end which is adapted to pierce through a layer of the compression dressing so that it may be connected to the external electrical generator located outside the dressing.

In further embodiments of the invention the portable treatment device may be configured to apply electrical signals between the electrodes according to the data recorded by the portable treatment device.

Referring to FIG. 11A a monitoring device 100 according to a tenth embodiment of the invention comprises a tilt and motion sensor 102, a memory 104, a display 106 and a housing 105. The tilt and motion sensor 102 and display 105 are substantially identical to the combined tilt and motion sensor and display, respectively, of the seventh embodiment.

Housing 105 will be described in more detail with reference to FIGS. 11B and 11C. Housing 105 is made of compressible foam and has a side wall 106 which is contoured to fit, in use, around part of the limb of a user. Under the influence of a force applied by a compression dressing 107 applied around the leg and the monitoring device the housing 105 fits against the limb 108 of a user as illustrated in FIG. 11C.

This device has the advantage that it provides a comfortable fit under a compression bandage and does not induce additional pain or cause secondary wounds. Further, the device does not impede the mobility of the wearer or interfere in their sleeping patterns Although in this embodiment the housing 105 is made of a foam-like material, in alternative embodiments the housing 105 may be made of any compressible elastomeric material.

In yet further embodiments the compression dressing may form part of the device.

In alternative embodiments of the monitoring device, the device may include a display for displaying data associated with the tilt and motion sensor, for example a display which is visible through a layer of compression dressing.

In further embodiments of the invention, the monitoring device may be disposed in a housing with only part of the housing being made of flexible material. For example the housing may be made up of two parts, a first part arranged in use to face the limb having a curved outer wall being made of compressible material and being adapted to fit around part of the limb, and a second part, arranged in use to face outwardly from a limb, the second part being made of rigid material

In a further embodiment of the invention a monitoring device may be mounted on a separate mount having a contoured side wall to fit around part of the limb of a user. The mount may be made of a compressible material such as rubber.

Referring to FIGS. 12, 13A and 13B, a a patient compliance monitor according to an eleventh embodiment of the invention comprises a housing 115, a pressure sensor 112, a memory 114 and a removal detector 117 for detecting removal of the monitoring device or a compression dressing from a limb of the patient. Housing 115 is similar to housing 105 of the tenth embodiment.

Removal detector 117 takes the form of a switch disposed on the concave wall 116 of the housing 115. In use, when the device 110 is fitted on the limb of a patient the skin of the patient pushes against the pressure button 117 depressing it towards the housing 115 such that an electrical circuit in the housing is opened indicating that the device is securely fitted on the leg or that a compression dressing is securely fitted around the leg and the device. If the device 110 is removed or the compression dressing is removed the switch 117 moves back in an outwardly direction from the device 110 and closes an electrical circuit so that an alarm to alert the user is activated. The activation is recorded in memory 114. The sense of activation of the switch may alternatively be reversed, with the circuit closed when the device is in place and open otherwise.

Thus the wearer is alerted if the device becomes loose or a compression dressing becomes lose and by recording removal data in the memory a healthcare practitioner can see that the treatment monitoring device has been removed during the course of treatment.

In alternative embodiments the switch may be replaced by any suitable pressure sensor which can detect the reduction in pressure when a device or a compression dressing is removed from the limb of a patient. In some embodiments the device may include a sole pressure sensor or switch for monitoring the presence of a medical appliance.

Such a device could be used to test the application of any medical appliance which may be inadvertently or prematurely removed by a patient or practitioner. Such appliances include, but are not limited to:
a. All forms of traditional dressings
b. Advanced dressings (e.g. silver)
c. Exudate devices (e.g. Kerraboot™)
d. Home-use and clinic-based Negative Wound Therapy Devices
e. All forms of energy devices providing, for example; electrical stimulation, heat therapy, diathermy, magnetic therapy, electromagnetic therapy, interferential, light therapy, shock-waves therapy
f. Pneumatic compression devices
g. Oxygen delivery devices
h. Bacteria/infection monitoring devices
i. Orthotic and prosthetic devices
j. Therapeutic pressure garments
k. Devices worn by patients in other areas of medicine where the premature removal of the device would impact health risks or the probability of a successful therapy.
l. Off-loading boot or shoe for treatment of diabetic foot or leg ulcers

It should be appreciated that the sensor may take any suitable form, such as any form of pressure sensor, any form of light sensor, or any other form of sensor that can detect the removal of a physical device attached to the body.

Detection of removal of the medical appliance may be based on the output of the measurement means. For example, in the embodiment shown in FIG. 7, the pressure sensor may be used to detect the presence of the patient compliance monitor or medical appliance such as compression dressing as well as to monitor pressure applied by a compression dressing.

It will be appreciated that any feature of the above described embodiments may be combined with one or more features of the other embodiments to provide a range of monitoring device which for monitoring a range of different physical parameters.

A method of monitoring treatment according to an embodiment of the invention is illustrated in FIG. 14 using the ambulatory monitor according to the fifth embodiment of the invention. Pads 62 and 64 incorporating pressure sensor 51 and temperature sensor 511 are placed in the region of a wound on the leg of a patient. Compression dressing 67 is placed over the pads 62 and 64. The pads are connected by wiring to housing 50 which is strapped to the lower leg of a patient close to the knee. In this way the pressure sensor 51 is able to detect if a sufficient pressure is maintained just above the ankle under a compression bandaging. This may be a strip to record pressure gradient. Housing 50 is positioned in such a way that display screen 55 is visible for reading data representative of the measure physical parameters.

Although the present invention has been described hereinabove with reference to specific embodiments, the present invention is not limited to the specific embodiments, and modifications will be apparent to a skilled person in the art which lie within the scope of the present invention.

1-153. (canceled)

154. A monitoring device for monitoring treatment of a wound or injured tissue on a limb of a person or an animal, the device comprising:
a monitoring arrangement to monitor at least one external physical parameter acting on the said limb, said at least one physical parameter having an influence on the healing of a wound or injured tissue on the said limb, the monitoring arrangement comprising at least one of a tilt sensor measure the tilt of said limb relative to the direction of gravitational force and a movement sensor to monitor motion of said limb; and
treatment delivery device to apply treatment for healing of the wound or injured tissue to the region of said wound, the treatment delivery device comprising a plurality of electrodes to apply electrical signals to the region of said wound or injured tissue and operable to deliver treatment according to the output of the monitoring arrangement.

155. A monitoring device according to claim 154, further comprising a memory to record data representative of the at least one external physical parameter.

156. A monitoring device according to claim 155, wherein the treatment delivery device is operable to apply treatment to the region of the wound or injured tissue according to data recorded by the memory.

157. A monitoring device according to claim 155, further comprising a data transmitter to transmit recorded data to an external device.

158. A monitoring device according to claim 154, wherein the monitoring arrangement further comprises at least one of a pressure monitor to monitor the pressure applied to a region of said wound or injured tissue and a temperature monitor to monitor the ambient temperature around said limb.

159. A monitoring device according claim 154, further comprising an alert device operable to generate an alert indicative of the status of the external physical parameter.

160. A monitoring device according to claim 154, wherein the treatment delivery device comprises a compression dressing arranged, in use, on a region of said limb to reduce pooling of blood in the region of said limb.

161. A monitoring device according to claim 160, further comprising a pressure sensor to detect a removal or reduction of pressure applied by a compression dressing in use.

162. A monitoring device according to claim 154, further comprising a detector to detect removal of the monitoring arrangement from said limb.

163. A monitoring device according to claim 154, where the tilt sensor comprises a first sensor for placement on said limb and a second sensor for placement on the patient's torso, the two sensors capable of assessing each others relative position in the vertical direction.

164. A method of monitoring treatment of a wound on the body of a person or an animal, the method comprising
monitoring at least one external physical parameter acting on the said body, said physical parameters having an influence on the healing of a wound or injured tissue on the said body and comprising at least one of measuring the tilt of a limb of said patient relative to the direction of gravitational force and monitoring motion of said limb; and
applying treatment for wound healing to the region of said wound or injured tissue by applying electrical signals to the region of said wound or injured tissue through a plurality of electrodes according to the output of the monitoring.

165. A method according to claim 164, further comprising recording data representative of the at least one external physical parameter in a memory.

166. A method according to claim 165, further comprising operating the treatment delivery device to apply treatment to the region of the wound or injured tissue according to data recorded by the memory.

167. A method according to claim 165, further comprising using a data transmitter to transmit data recorded in the memory to an external device.

168. A method according to claim 164, wherein monitoring the at least one external physical parameter further comprises at least one of measuring the pressure applied to a region of said body and monitoring the ambient temperature around said body.

169. A method according to claim 164, further comprising arranging a compression dressing on a region of said limb to reduce pooling of blood in the region of said limb.

170. A method according to claim 169, wherein monitoring the at least one external physical parameter further comprises measuring the pressure applied to a region of said body by a compression dressing.

171. A method according to claim 164, further comprising detecting removal of the monitoring arrangement from said limb.

172. A method according to claim 164, further comprising measuring tilt using a first sensor placed on said limb and a second sensor placed on the patient's torso, the two sensors capable of assessing each others relative position in the vertical direction.

173. A monitoring device for monitoring treatment of a wound or injured tissue on a limb of a person or an animal, the device comprising:
monitoring arrangement comprising a tilt sensor to measure the tilt of said limb relative to the direction of gravitational force;
treatment delivery device to apply treatment for healing of the wound or injured tissue to the region of said wound.

174. A monitoring device for monitoring treatment of a wound or injured tissue on a limb of a person or an animal, the device comprising
a monitoring arrangement to monitor at least one external physical parameter acting on the said limb, said at least one physical parameter having an influence on the healing of a wound or injured tissue on the said limb;
a memory to record, over a period of time, data representative of said at least one physical parameter; and
treatment delivery device to apply treatment for healing of a wound or injured tissue to the region of said wound or injured tissue, the treatment delivery device comprising a plurality of electrodes to apply electrical signals to the region of said wound or injured tissue and is operable to apply treatment according to the recorded data.

175. A monitoring device according to claim 174, further comprising a data transmitter to transmit recorded data to an external device.

176. A monitoring device according to claim 174, wherein the monitoring arrangement further comprises at least one of a pressure monitor to monitor the pressure applied to a region of said wound or injured tissue and a temperature monitor to monitor the ambient temperature around said limb.

177. A monitoring device according claim 174, further comprising an alert device operable to generate an alert indicative of the status of the external physical parameter.

178. A monitoring device according to claim 174, wherein the treatment delivery device comprises a compression dressing arranged, in use, on a region of said limb to reduce pooling of blood in the region of said limb.

179. A monitoring device according to claim 178, further comprising
a pressure sensor to detect a removal or reduction of pressure applied by the compression dressing in use.

180. A monitoring device according to claim 174, further comprising a detector to detect removal of the monitoring arrangement from said limb.

181. A monitoring device according to claim 174, further comprising a tilt sensor comprising a first sensor for placement on said limb and a second sensor for placement on the patient's torso, the two sensors capable of assessing each others relative position in the vertical direction.

182. A patient compliance monitor for monitoring compliance of a patient to a treatment regime for treatment of a medical condition, the patient compliance monitor comprising
a sensor device comprising a tilt sensor to measure the tilt of said limb relative to the direction of gravitational force, the tilt sensor comprising a first sensor for placement on said limb and a second sensor for placement on the patient's torso, the two sensors capable of assessing each others relative position in the vertical direction;
recording device to record over a period of time data representative of measured tilt; and
comparing device to compare the recorded data with data indicative of the treatment regime to determine patient compliance to the treatment regime.

183. A monitoring device for monitoring treatment of a wound or injured tissue on a limb of a person or an animal, the device comprising
monitoring device comprising a tilt sensor to measure the tilt of said limb relative to the direction of gravitational force, the tilt sensor comprising a first sensor for placement on said limb and a second sensor for placement on the patient's torso, the two sensors capable of assessing each others relative position in the vertical direction; and
treatment delivery device to apply treatment for healing of the wound or injured tissue to the region of said wound or injured tissue.

184. A monitoring device for monitoring treatment of a wound or injured tissue on a limb of a person or an animal, the device comprising
monitoring means for monitoring at least one external physical parameter acting on the said limb, said at least one physical parameter having an influence on the healing of a wound or injured tissue on the said limb, the monitoring means comprising et least one of a tilt sensor for measuring the tilt of said limb relative to the direction of gravitational force and a movement sensor for monitoring motion of said limb; and
treatment delivery means for applying treatment for healing of the wound or injured tissue to the region of said wound, the treatment delivery means comprising a plurality of electrodes for applying electrical signals to the region of said wound or injured tissue and operable to deliver treatment according to the output of the monitoring means.

185. A monitoring device for monitoring treatment of a wound or injured tissue on a limb of a person or an animal, the device comprising:
monitoring means comprising a tilt sensor for measuring the tilt of said limb relative to the direction of gravitational force;
treatment delivery means for applying treatment for healing of the wound or injured tissue to the region of said wound.

186. A monitoring device for monitoring treatment of a wound or injured tissue on a limb of a person or an animal, the device comprising
monitoring means for monitoring at least one external physical parameter acting on the said limb, said at least one physical parameter having an influence on the healing of a wound or injured tissue on the said limb;
recording means for recording, over a period of time, data representative of said at least one physical parameter; and
treatment delivery means for applying treatment for healing of a wound or injured tissue to the region of said wound or injured tissue, the treatment device comprising a plurality of electrodes for applying electrical signals to the region of said wound or injured tissue and is operable to apply treatment according to the recorded data.

187. A patient compliance monitor for monitoring compliance of a patient to a treatment regime for treatment of a medical condition, the patient compliance monitor comprising
measurement means comprising a tilt sensor for measuring the tilt of said limb relative to the direction of gravitational force, the tilt sensor comprising a first sensor for placement on said limb and a second sensor for placement on the patient's torso, the two sensors capable of assessing each others relative position in the vertical direction;
recording means for recording over a period of time data representative of measured tilt; and
comparative means for comparing the recorded data with data indicative of the treatment regime to determine patient compliance to the treatment regime.

188. A monitoring device for monitoring treatment of a wound or injured tissue on a limb of a person or an animal, the device comprising
monitoring means comprising a tilt sensor for measuring the tilt of said limb relative to the direction of gravitational force, the tilt sensor comprising a first sensor for placement on said limb and a second sensor for placement on the patient's torso, the two sensors capable of assessing each others relative position in the vertical direction; and
treatment delivery means for applying treatment for healing of the wound or injured tissue to the region of said wound or injured tissue.
